(19) United States
(12) Patent Application Publication
(10) Pub. No.: US 2025/0083399 A1
(43) Pub. Date: Mar. 13, 2025

(54) A COMPRESSION MOULDED BODY

(71) Applicant: INVIBIO DEVICE COMPONENT MANUFACTURING LIMITED, Thornton Cleveleys, Lancashire (GB)

(72) Inventors: Fedra Zaribaf, Thornton Cleveleys, Lancashire (GB); Stephen Bonham, Thornton Cleveleys, Lancashire (GB)

(21) Appl. No.: 18/728,665

(22) PCT Filed: Jan. 11, 2023
(86) PCT No.: PCT/GB2023/050045

§ 371 Date: Jul. 12, 2024

(30) Foreign Application Priority Data
Jan. 13, 2022 (GB) 2200415.4

(51) Int. Cl. B29C 70/46; B29C 43/02; B29C 43/36; B29K 71/00; B29K 105/08; B29K 307/04; B29L 31/00; B29L 31/30

(52) U.S. Cl. CPC B29C 70/465; B29C 43/02; B29C 43/36; B29K 2071/00; B29K 2105/0881; B29K 2307/04; B29L 2031/3076; B29L 2031/7532

(57) ABSTRACT

The invention relates to a method of manufacturing a body, the method comprising: forming a plurality of layers (301) of composite material comprising reinforcement fibre and polyaryletherketone, a first layer (301) of the plurality of layers (301) being shaped such that in a first region it is narrower along a first axis than a second layer (301); stacking the plurality of layers (301) within a mould cavity (905) of a mould tool (900) such that the first layer (301) defines a first side of the stack and the second layer (301) is within the stack; and compression moulding the stacked plurality of layers (301) within the mould cavity (905); wherein the mould cavity (905) is defined by at least one surface of the mould tool (900) shaped to align the slacked plurality of layers (301) in the first region such that a resulting compression moulded body is thinner in the first region than in a second region.

### TECHNICAL FIELD

The present invention relates to a compression moulded body, and further relates to a method of manufacturing a compression moulded body and a mould tool for forming the compression moulded body. Certain examples of the present invention relate to a bone fracture plate formed as a compression moulded body.

### BACKGROUND

It is known to manufacture parts by compression moulding a plurality of layers of a polymer material to form a compression moulded body. The end result may be referred to as a laminate structure. Typically, each layer (also referred to as a laminate or ply) may comprise a composite material comprising a polymer and reinforcement fibres. An example of such a composite material comprises carbon fibre and polyaryletherketone (PAEK). The polyaryletherketone may comprises polyetheretherketone (PEEK).

Polymer compression moulding may be used in a range of applications to form laminated products, including, for example, in the manufacture of medical devices such as orthopaedic implants. For example, a bone fracture plate (used to repair bone trauma) may be formed by compression moulding a plurality of layers of polymer (with or without reinforcement fibres). Conventionally, a mould tool is used having a mould cavity defining a shape of a blank part. The mould cavity is then filled with layers that are cut or otherwise shaped to fill the mould cavity, the mould cavity is closed off and the mould cavity is compressed and heated to form a compression moulded blank. Typically, the edges of the moulded blank are then machined down to a nominal (intended) part size for the final product. There may be further post-moulding processes. For instance, in the case of a bone fracture plate, screw holes may be milled through the plate. In the present specification bone fracture plates are presented as an example of compression moulded body, however the present invention is not limited to this. The skilled person will understand that the same compression moulding process may be used to form parts in a wide range of industries including the automotive and aerospace sectors.

For implantable medical devices the use of composite polymeric materials may be advantageous over conventional uses of metal because flexibility can be provided to the device while maintaining the strength required for load bearing. In addition, for a bone fracture plate, polymeric materials may be less likely to cause bone deterioration.

Current techniques for manufacturing a compression moulded part may not give the flexibility or accuracy required for certain applications.

It is an aim of certain examples of the present invention to solve, mitigate or obviate, at least partly, at least one of the problems and/or disadvantages associated with the prior art. Particularly, certain examples of the present invention aim to provide a compression moulded body with improved properties and/or component accuracy.

### BRIEF SUMMARY OF THE INVENTION

According to a first example of the present invention there is provided a method of manufacturing a body, the method comprising: forming a plurality of layers of composite material comprising reinforcement fibre and polyaryletherketone, a first layer of the plurality of layers being shaped such that in a first region it is narrower along a first axis than a second layer; stacking the plurality of layers within a mould cavity of a mould tool such that the first layer defines a first side of the stack and the second layer is within the stack; and compression moulding the stacked plurality of layers within the mould cavity; wherein the mould cavity is defined by at least one surface of the mould tool shaped to align the stacked plurality of layers in the first region such that a resulting compression moulded body is thinner in the first region than in a second region.

According to a further example of the present invention there is provided a compression moulded body comprising: a plurality of stacked layers of composite material comprising reinforcement fibre and polyaryletherketone; wherein in a first region of the body a first layer defining a first side of the stack is narrower along a first axis than a second layer within the stack such that the body is thinner in the first region than in a second region.

According to a further example of the present invention there is provided a mould tool for forming a compression moulded body from a plurality of stacked layers of composite material comprising reinforcement fibre and polyaryletherketone, the mould tool comprising: a mould cavity configured to receive the stacked layers, a first layer defining a first side of the stack of the plurality of layers being shaped such that in a first region it is narrower along a first axis than a second layer within the stack; wherein the mould cavity is defined by at least one surface of the mould tool shaped to align the slacked plurality of layers in the first region such that a resulting compression moulded body is thinner in the first region than in a second region.

An advantage of certain examples of the present invention is that the compression moulded body may suffer from less warpage compared to compression moulded bodies formed according to conventional techniques. Furthermore, a new type of compression moulded body may be designed and produced more quickly with less recourse to trial and error in its design. In some examples the mould cavity of a mould tool may be shaped to align a stacked plurality of layers during compression moulding in order to form a compression moulded body with these advantageous properties.

### BRIEF DESCRIPTION OF THE DRAWINGS

Examples of the invention are further described hereinafter with reference to the accompanying drawings, in which:

FIG. 1 is a schematic drawing of a lay-up arrangement that may be used in the manufacture of a compression moulded body;

FIG. 2 is a flow chart illustrating a method of manufacturing of a compression moulded body;

FIGS. 3a to 3g show different steps in the method of manufacturing of FIG. 2;

FIG. 4 is an exploded perspective view of a mould tool according to the prior art;

FIG. 5 is a schematic side view of a medical device according to the prior art;

FIG. 6 is a schematic sectional side view of a part of the medical device of FIG. 5;

FIG. 7 is a perspective view of part of a compression moulded body according to an example of the present invention;

FIG. 8 is a side view of part of the compression moulded body of FIG. 7;

FIG. 9 is an exploded perspective view of a mould tool for forming a compression moulded body according to an example of the present invention;

FIG. 10 is a sectional view of part of the mould tool of FIG. 9;

FIG. 11 is a perspective view of part of a moulded blank formed using the mould tool of FIGS. 9 and 10;

FIG. 12 is a perspective view of a machined compression moulded body formed from a moulded blank according to FIG. 11, including an enlarged section; and

FIG. 13 is a series of images showing each ply forming the moulded blank of FIG. 11.

### DETAILED DESCRIPTION

The present invention relates to a compression moulded body and a method of manufacturing a compression moulded body. Examples of the present invention presented below relate particularly to the design and manufacture of a compression moulded bone fracture plate, however the present invention is more widely applicable than this, and also relates to a compression moulded body for use in, for example, aircraft parts, including aircraft brackets, and other load bearing parts

A compression moulded body is formed from a plurality of layers of polymer. The layers may be formed from a composite material. The composite material forming the layers may be provided in the form of a tape. Layers (also referred to as “plies” or “laminates”) may be formed of joined portions of tape. The composite material may comprise polyaryletherketone as an example polymer and reinforcement fibre, for instance carbon fibre. The polyaryletherketone may suitably be polyetheretherketone (PEEK). Greater detail regarding these materials is given in the description below.

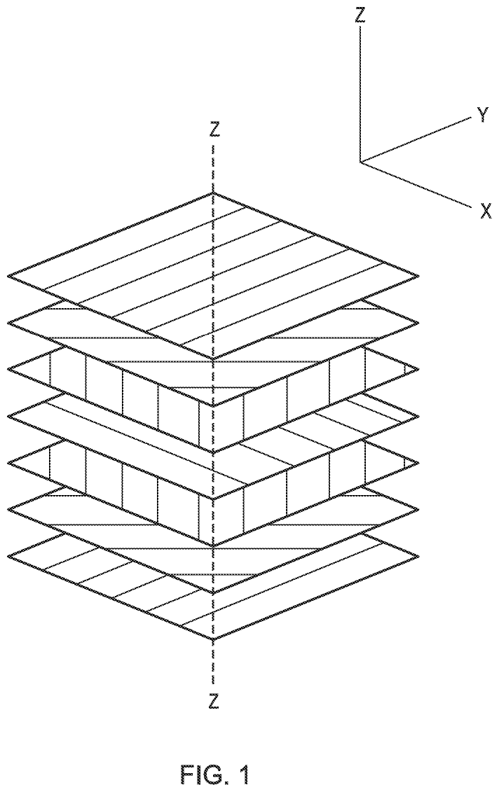

Referring to FIG. 1, this is a schematic drawing of an example lay-up arrangement of layers of tape that may be compression moulded using a mould tool according to an example of the present invention to form a compression moulded body. The layers are stacked into a mould cavity in the Z direction, each layer comprising a composite tape layer (or ply) having varying orientations of carbon fibres in an X-Y plane. Starting from the bottom of the lay-up as shown, the first layer is formed of tape that is unidirectionally aligned along the Y-axis (0°). The second layer is formed of tape that is unidirectionally aligned at 45° to the axis of the first layer. The third layer is formed of tape 14c that is unidirectionally aligned at −45° to the axis of the first layer. The fourth layer is formed of tape 14d that is unidirectionally aligned at 90° to the axis of the first layer (that is, along the X-axis). The pattern is repeated so that the overall structure has the following alignment: 0°, 45°, −45°, 90°, −45°, 45° and 0. The resulting laminate may be compression moulded under heat and pressure to form a compression moulded semi-finished component—a blank. Additional features such as holes can then be machined into the blank and edge regions of the blank machined to size to form a finished compression moulded body.

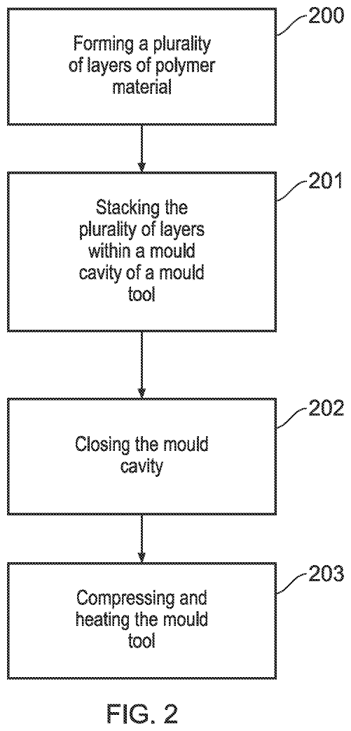

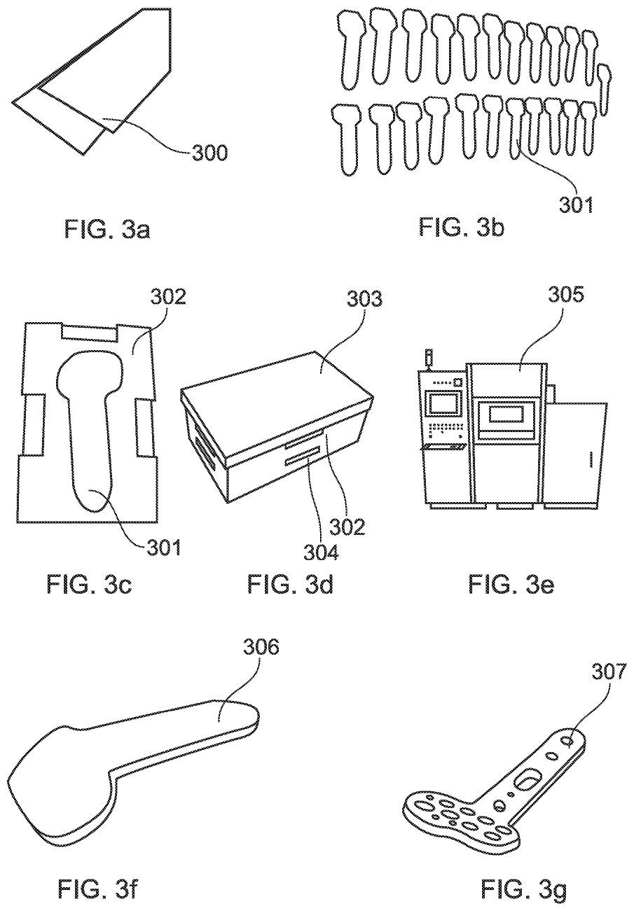

Referring now to FIGS. 2 and 3a to 3g a method of manufacturing a compression moulded body will now be described. The method begins at step 200 with the formation of a plurality of layers of polymer material. As noted previously, this may suitably comprise a composite material including a polymer and reinforcement fibres. This may be provided as a tape 300 as shown in FIG. 3a. The tape 300 may be cut or otherwise shaped to form a plurality of layers 301 (also referred to as plies) as shown in FIG. 3b. It can be seen that in this example each layer 301 is substantially the same shape. Two or more pieces of tape 300 may be joined to form layers 301, if required.

At step 201 the plurality of layers 301 are stacked within a mould cavity of a middle section 302 of a mould tool as shown in FIG. 3c. This may be referred to as a ply layup. As discussed in connection with FIG. 1, for composite plies each ply may be cut such that the fibres extend in a predetermined direction.

At step 202 the mould tool is closed, by sandwiching the middle section 302 between a top section 303 and a bottom section 304 as shown in FIG. 3d. Closing the mould tool seals the layers 301 within the mould cavity.

At step 203 the mould tool is heated and compressed, with FIG. 3e showing a suitable apparatus for heating and applying compressive force to the mould tool. This compresses the layers 301 and bonds them together as the polymer softens or melts. When the mould tool is opened a blank part 306 is removed as shown in FIG. 3f. The shape of the blank part is defined by the shape of the mould cavity.

FIG. 3g shows the end product, in this case a bone fracture plate 307, after various post-moulding processes have been applied (for instance machining down the edges of the blank 306 and milling screw holes.

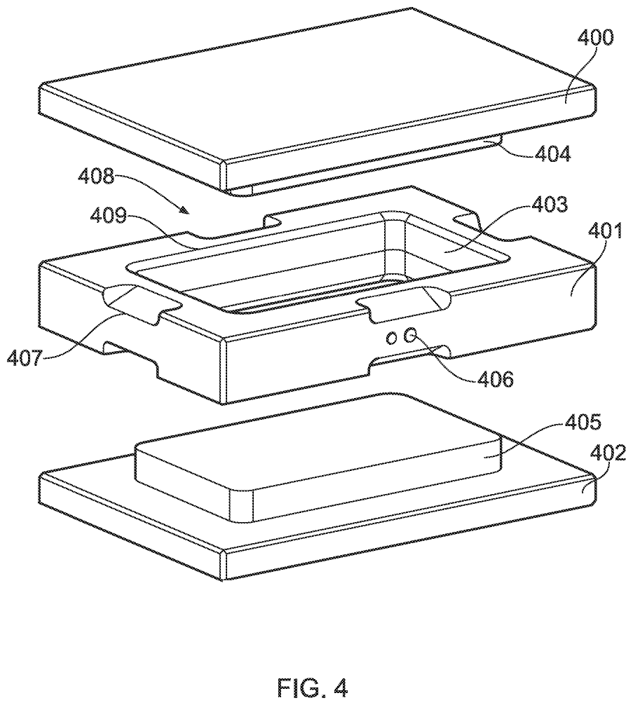

Referring now to FIG. 4, a conventional mould tool will now be described. The mould tool comprises a top section 400, a middle section 401 and a bottom section 402 that collectively define a mould cavity 408. A mould opening 409 extends through the middle section 401 and is defined by a side wall 403. The side wall 403 defines the edges of the mould cavity 408. The top section 400 includes a protrusion 404 that is shaped to fit into an upper end of the mould opening 409. Similarly, the bottom section 402 includes a protrusion 405 that is shaped to it into a lower end of the mould opening 409. The protrusions 404, 405 define upper and lower sides or surfaces of the mould cavity 408. The fit between the mould opening 409 and the protrusions 404, 405 is selected to be close so that when joined together the protrusions are in touching contact with the side wall 403 to substantially close off the mould cavity. In some examples the middle section 401 and bottom section 402 may be integrally formed. However, providing them separately can make it easier to remove a blank part. FIG. 4 further shows one or more thermocouple holes 406 for temperature sensing during moulding and chamfers 407 distributed about the upper and lower edges of the middle section 401 to assist the user in grasping the top and bottom sections 400, 402 when assembling or disassembling the mould tool.

To operate the mould tool the middle and bottom sections 401, 402 are coupled together to close off the bottom of the moulding opening 409 such that a mould cavity 408 with a closed base is defined. The layers 301 may then be stacked in the mould cavity 408 as described above and the mould cavity closed by coupling the top section 400. The mould tool may be a floating mould tool in which a gap is preserved between opposed surfaces of the top section 400 and the middle section 401. A floating mould tool ensures that all compressive force applied to the mould tool is transferred to the stacked layers 301 within the mould cavity 408 by preventing the mould tool from bottoming out.

Referring back to the layup arrangement of FIG. 1, it is known that when building such an arrangement consideration must be given to the symmetrical nature of the stack to minimise bending or warping. However, when it is required to impart a varying cross-section—that is, depth along the Z axis—to the resulting compression moulded part, extra care is needed to achieve the desired thickness at specific locations. Typically, this is achieved by a graduated layered arrangement whereby smaller lengths of plies are placed in a stepped sequence on top of longer lengths of ply. To prevent shearing or delamination at the exterior facing surfaces due to graduated exposed edges, the graduated layered arrangement is covered over by an outer ply forming substantially the whole of the top or bottom surface of the body. That is, the graduated exposed edges are buried such that top and bottom surfaces of the compression moulded body provide a continuous, unbroken surface. Edges of the individual ply are exposed only at the sides of the compression moulded body, which typically are machined after moulding a blank part to reduce the blank to the nominal part size. This serves to reduce rough edges, which is of particular importance for medical applications. Accordingly, the unbroken top and bottom surfaces of the compression moulded body minimise tissue contact with composite filler.

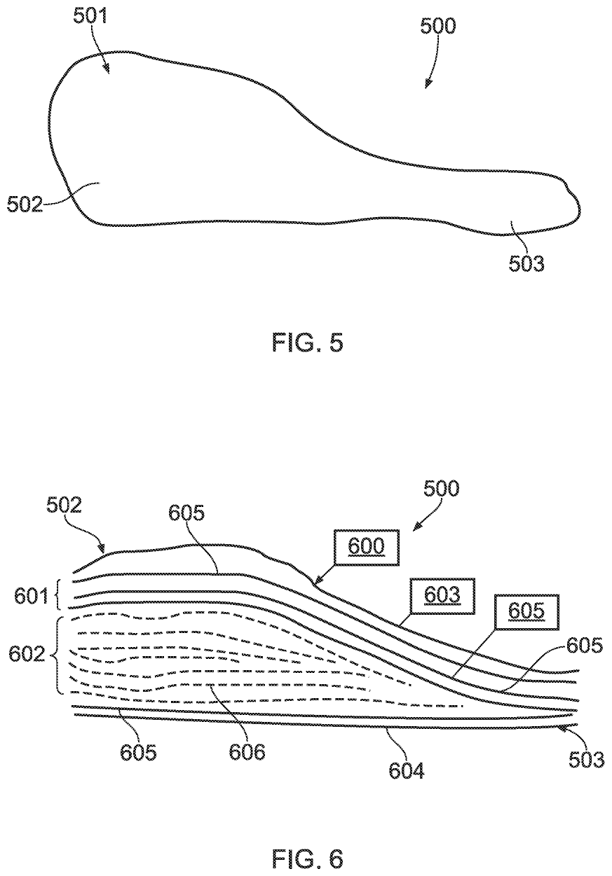

Graduated layered plies may be referred to as filler layers and are substantially or wholly encapsulated by the continuous outer plies. An example of an implantable medical device formed in this way will now be presented with reference to FIGS. 5 and 6. FIG. 5 is a schematic side view of an implantable medical device 500. The device 500 is an implantable medical device that comprises a body 501 having a head 502 and a tail 503. It can be seen that the cross sectional area of the device 500 varies along the longitudinal length of the body 501, with the head 502 being much greater in thickness compared to the tail 503.

Turning to the sectional side view of FIG. 6, the device 500 comprises a first portion 600, a packing portion 601 and an insert 602. The first portion 600 includes a first ply 603 and a second ply 604. The first and second plies 603, 604 together form exterior top and bottom surfaces of the device 500. The packing portion 601 comprises a plurality of packing plies 605 which are adjacent an inner face of one or both of the exterior surface plies 603, 604. As shown in FIG. 6, the packing plies 605 are located either side of the insert 602, and adjacent the first and second plies 603, 604. The packing plies 605 are symmetrical about the longitudinal axis of the body 4. In an alternative embodiment, packing plies are provided between insert plies.

The insert 602 comprises at least one insert ply 606. In the embodiment shown in FIG. 6, a plurality of insert plies 606 (represented by dashed lines) is provided in the device 500. The or each insert ply 606 is shorter in length when compared to the packing plies 605. In so doing, the packing plies 605 surround the insert 602, encasing it within the packing portion 601.

The present inventors have identified that a compression moulded body according to FIGS. 5 and 6 whereby a thickness variation is achieved by burying an insert 602 underneath exterior layers 603, 604 and optionally packing plies 605 may suffer from certain disadvantages. The outer exterior layers 603, 604 may suffer from wrinkling or stretching where they accommodate thickness variations for the device 500. Wrinkling or stretching may be particularly acute where the thickness of device 500 changes owing to insert 602 occur within a short distance along a surface of the moulded body. Wrinkling or stretching may result in an uneven outer surface. Furthermore, wrinkling or stretching of a ply may resulting in the moulded body warping. This warping may occur as the compression moulded body cools after moulding. Further warping may occur in use, during the lifespan of the moulded body. For applications where an accurate shape is required, including for a bone fracture plate, warpage may result in the part being unusable.

Where warpage is found to occur, it may be that refinements can be made to the design of the compression moulded body to minimise warpage. For instance, the size and shape of an insert or the orientations of one or more ply may be changed. It will be appreciated that even where warpage can be reduced to acceptably low levels, this process of trial an error may be time consuming and expensive.

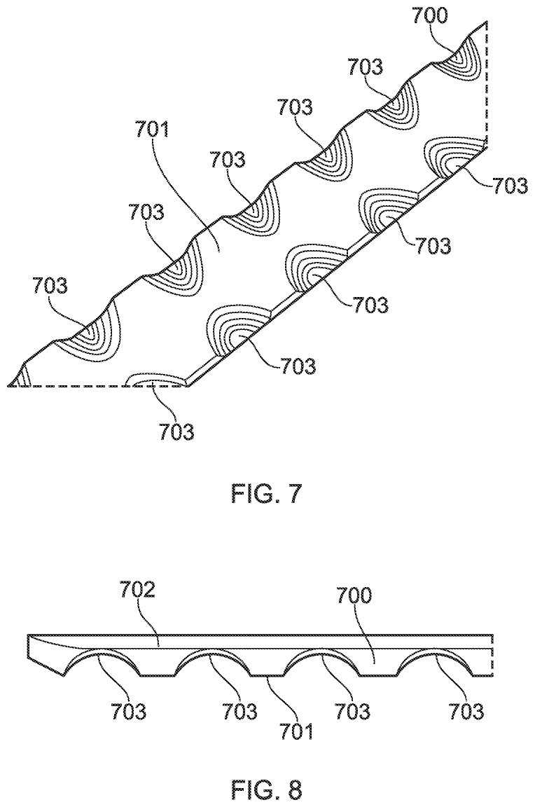

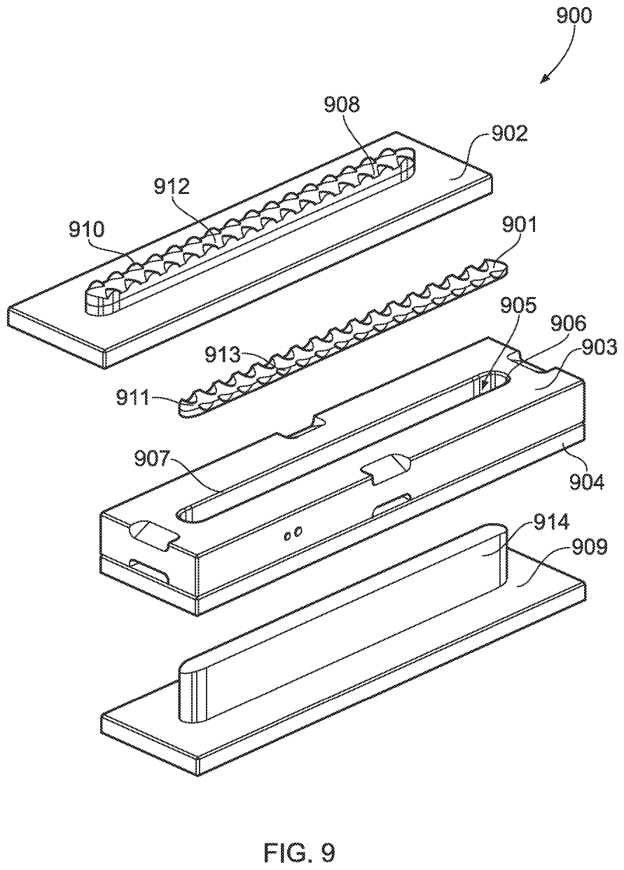

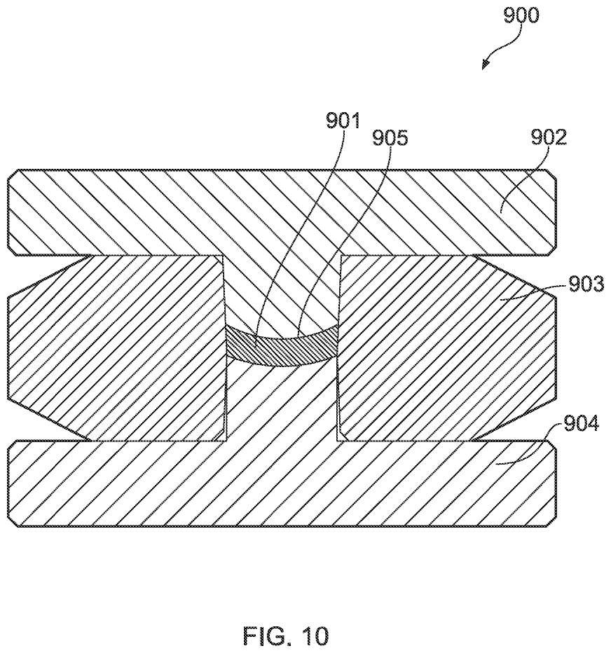

FIG. 7 is a perspective view of part of a compression moulded body, particularly a moulded blank, according to an example of the present invention. FIG. 8 is a side view of part of the compression moulded body of FIG. 7. In the example of FIGS. 8 and 9 the compression moulded body is an elongate bone fracture plate 700. FIG. 7 shows the bone facing surface 701 of the plate 700 facing upwards and FIG. 8 reveals both the bone facing surface 701 and the opposite tissue facing surface 702. In use the bone fracture plate 700 may be applied to a patient's bone bridging a fracture. Screw holes may be provided in the bone fracture plate 700 to secure the plate 700 to the bone. FIGS. 7 and 8 illustrate moulded blanks prior to the milling of screw holes and further machining to reduce the moulded part to the nominal part size.

The compression moulded body may be an aircraft part having all or any one of the features hereinafter described in relation to the bone plate.

It can be seen that the bone facing surface 701 is concave and the tissue facing surface 702 is convex. This permits better conformance to the shape of a bone. The curved form of the bone plate is achieved through compression moulding flat laminate layers within a mould tool having a curved mould cavity. The bone facing surface 701 includes one or more undercuts 703 (also referred to as cut outs, scallops or recesses). The undercuts 703 locally reduce the thickness of the bone fracture plate 700, as can best be seen in FIG. 8. The undercuts 703 in the example of FIGS. 7 and 8 are arranged along both long edges of the plate 700, however in other examples they may be only on one side or only a single undercut may be provided. FIG. 7 shows an example in which the undercuts 703 are provided in pairs and evenly spaced along both long edges of the plate.

The undercuts 703 serve to increase the flexibility of the bone fracture plate 700 along its longitudinal axis, as is conventional for similar bone fracture plates formed from metals. It will be appreciated that longitudinal flexibility for the bone fracture plate 700 may be further controlled by appropriate selection and arrangement of the plies and their orientations, as discussed above in connection with FIG. 1. The undercuts 703 also reduce contact between the bone plate 700 and the bone which may be desirable to reduce damage or irritation to the bone. While the undercuts 703 do include a rougher surface than the surrounding single layer portion of the bone facing surface 701, this rougher surface is less significant in terms of biocompatibility on a bone facing surface than it would be on a tissue facing surface. Additionally, the design of the undercuts means that the surface of each undercut does not fully contact the underlying bone.

It can be seen in FIG. 7 that within each undercut 703 the edges of each layer are revealed. In particular, a first layer which is outermost in the stack on the bone facing surface 701 is narrower in a first region (the region of an undercut) than one or more underlying layer. FIG. 7 shows a particular example in which in the region of an undercut 703 each layer is wider as you move down the stack from the bone facing surface 701: a series of layer edges is revealed. The undercuts 703 in the example of FIG. 7 do not go all the way through the thickness of the bone plate 700, but within a portion of stacked layers each layer may be progressively wider as the stack is traversed from the bone facing surface 701 to the tissue facing surface 702. However, this progressive widening is not essential: there may be two or more layers in an undercut that are the same width, and both narrower than a lower layer (further from the bone facing surface 701).

It will be appreciated that the bone fracture plate 700 of FIGS. 7 and 8 in the region of the undercuts 703 is analogous the to the thickness variation imparted by insert 602 in the device 500 of FIGS. 5 and 6, differing in that the graduated edges in the undercuts are not buried under packing plies or exterior plies. Advantageously, because no single layer is stretched across an undercut upon the bone facing surface 701, the problem of wrinkling or stretching described above in connection with FIGS. 5 and 6 does not apply. It can be seen in FIGS. 7 and 8 that each layer forming the compression moulded plate has substantially the same curved form, differing only in the width across the layer in the region of the undercut. Table 1 presented below shows measurements for a series of measurement locations along the length of the bone plate 700 of FIGS. 7 and 8. The inner radius refers to a measurement (in millimetres) of the radius of curvature of the bone facing surface 701 and the outer radius refers to a measurement (in millimetres) of the radius of curvature of the tissue facing surface 702 at a sample point along the length of the bone plate. It can be seen that the standard deviation for both measured radii is low, indicating that a low degree of warpage is present along the length of the bone plate 700.

TABLE 1

| | Location | Location | Location | Location | Location | | Standard |
|---|---|---|---|---|---|---|---|
| | 1 | 2 | 3 | 4 | 5 | Average | Deviation |
| Inner | 11.57 | 11.62 | 11.62 | 11.62 | 11.56 | 11.598 | 0.0247656 |
| radius | | | | | | | |
| Outer | 14.53 | 14.51 | 14.63 | 14.53 | 14.50 | 14.54 | 0.0424264 |
| radius | | | | | | | |

According to certain examples of the present invention, a compression moulded body can be formed with thickness variations formed from layers having different widths using a suitable mould tool having a mould cavity that includes at least one surface shaped to align a stacked plurality of layers with differing shapes (unlike the uniformly shaped layers of FIG. 3b). Referring now to FIGS. 9 and 10 a mould tool 900 according to an example of the present invention for use in compression moulding a blank part 901 will now be described. Similar to FIG. 4, mould tool 900 tool comprises a top section 902, a middle section 903 and a bottom section 904 that collectively define a mould cavity 905. It can be seen from the cross section of FIG. 10 that the mould cavity is generally curved such that a curvature is applied to the resulting moulded blank along its length to conform to a bone surface. A mould opening 906 extends through the middle section 903 and is defined by a side wall 907. The side wall 907 defines the edges of the mould cavity 905.

The top section 901 includes a protrusion 908 that is shaped to fit into an upper end of the mould opening 906. Similarly, the bottom section 904 includes a protrusion that is shaped to it into a lower end of the mould opening 409, though in FIG. 9 the middle section 903 and the bottom section 904 are shown coupled together so the protrusion is not directly visible. FIG. 9 further shows a ejection tool 909 generally corresponding to the bottom section 904 although including a taller protrusion 914 such that when the bottom section 904 is removed the ejection tool 909 can be inserted into the mould opening 906 to release a moulded blank 901 from the mould cavity 905. The protrusions 908, 909 define upper and lower sides or surfaces of the mould cavity 905. The fit between the mould opening 906 and the protrusions 908, 908 is selected to be close so that when joined together the protrusions are in touching contact with the side wall 907 to substantially close off the mould cavity 905. In some examples the middle section 903 and bottom section 904 may be integrally formed. However, providing them separately can make it easier to remove a blank part using the ejection tool 909.

To operate the mould tool the middle and bottom sections 903, 904 are coupled together to close off the bottom of the moulding opening 906 such that a mould cavity 905 with a closed base is defined. Shaped layers may then be stacked in the mould cavity 905 as described above and the mould cavity closed by coupling the top section 902. The mould tool 900 may be a floating mould tool in which a gap is preserved between opposed surfaces of the top section 902 and the middle section 903. A floating mould tool ensures that all compressive force applied to the mould tool is transferred to the stacked layers in the mould cavity 905. Once the mould cavity 905 is closed, the mould tool is compressed to apply compression to the stacked layers in the mould cavity 905 at the same time as they are heated.

As previously noted in at least one region the width of at least one layer may differ to define a thickness variation for the moulded blank in which at least a top or bottom surface is narrower than a layer within a middle portion of the stack. According to examples of the present invention the mould cavity is shaped so as to ensure that the shaped layers are correctly aligned to form thickness variations. In the example of FIGS. 9 and 10 the protrusion 908 of the top section 902 is shaped with rounded portions 910 that correspond to the undercuts 911 in the formed blank 901 (only one of which is identified). That is, the end face 912 of the protrusion 908 is a corresponding reverse image to the bone facing surface 913 of the moulded blank 901. Any misalignment of layers within the mould cavity 905 is corrected by the shape of end face 912. It will be appreciated that alignment surfaces or shapes can be provided on any internal surface of the mould cavity as required to correctly align shaped layers.

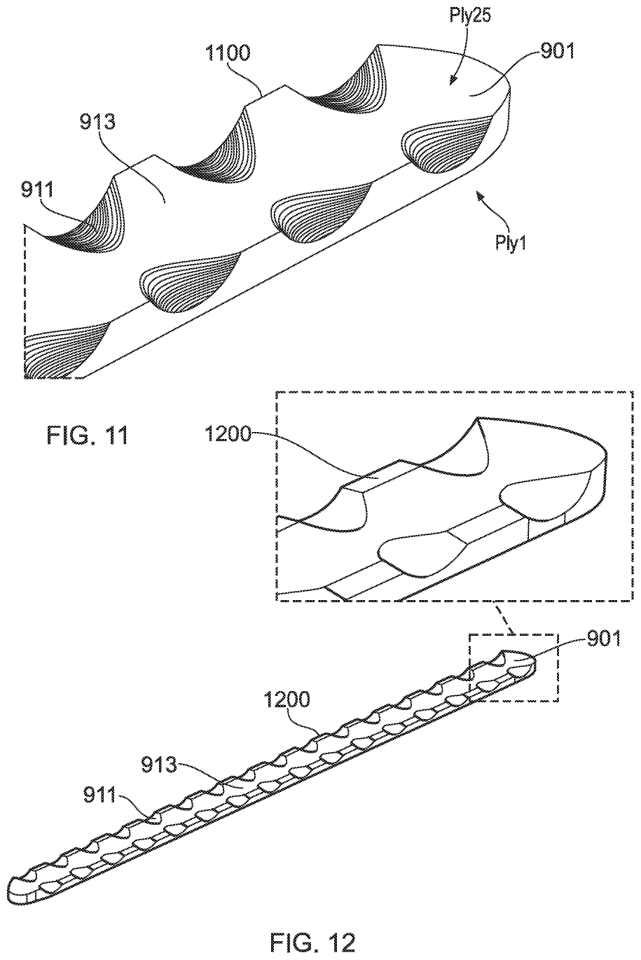

FIG. 11 is an enlarged view of part of the moulded blank 901 of FIG. 9 revealing the bone facing surface 913 and undercuts 911. In each undercut, the edges of the layers of plies exposed by the undercut can be seen. It can be seen that between each undercut 911 edge portions of the bone facing surface 913 extend to the full width of the moulded blank. This form protrusions 1100. In some examples, as illustrated in FIG. 12, particularly in the enlarged portion, the moulded blank is machined to remove protrusions 1100, forming instead flattened surfaces 1200. It will be appreciated that instead the layers forming upper portions of the moulded blank 901 may be appropriately shaped to impart the flattened surfaces 1200. However, in some examples it has been found to improve the moulding process to ensure that between undercuts 911 the layers are uniformly the same width so as to make it easier for end face 912 of the top section of the mould tool to align the layers within the mould cavity 905. At the same time further machining may be performed to edges of the moulded blank 901 to reduce the blank to the nominal part size for the bone fracture plate. Screw holes may also be milled, though this is not shown in FIG. 12.

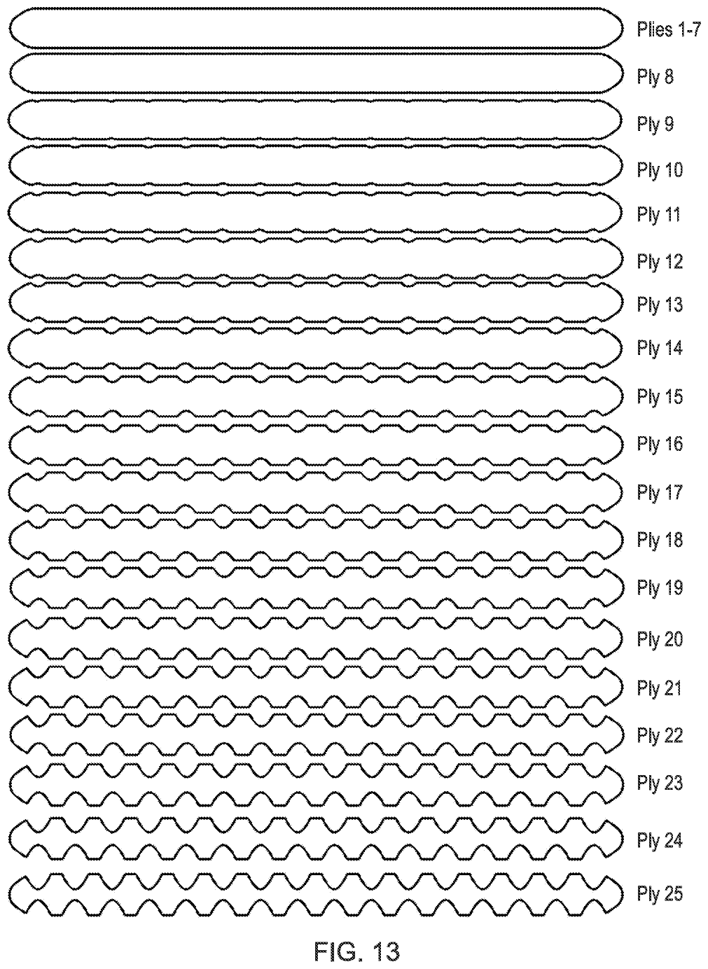

FIG. 13 provides a series of images showing the shape of each layer of ply forming the moulded blank of FIG. 11. FIG. 11 indicates that ply 1 is the bottom layer and ply 25 is the top layer forming the bone facing surface 913. Plies 1 to 7 have the same shape (with no undercut formation) and so only a single image is given. Each of ply 8 to ply 25 includes a progressively larger cut out. As explained previously, in other examples of the invention, two or more of plies 8 to 25 may have the same shape. In the example of FIG. 13, once stacked in the correct order within the mould cavity 905 of mould tool 900, and subjected to compression moulding, a moulded blank according to FIG. 11 will be formed.

The shape of protrusion 90 forming part of top section 902 serves to ensure correct alignment of the layers.

In examples of the present invention described above undercuts or scallops, or indeed any arbitrary thickness variation for a compression moulded part (where the thickness variation extends to an outer surface) may be formed by varying the shape of at least one layer within the stack and then shaping a mould tool to ensure that the layers are correctly aligned. However, in an alternative example, a compression moulded part such as bone fracture plate may be formed within a uniform thickness and then top or bottom surfaces machined to impart a thickness variation. It will be appreciated that the end result may be substantially the same: a varying thickness compression moulded part in which the top or bottom layer is narrower than an underlying layer.

It will be appreciated that the compression moulding method to form a bone fracture plate in the preceding is the same as for that given in FIG. 2, differing only that each layer in the plurality of stacked layers may have a different shape and that the mould tool is shaped to ensure that the differing layers remain correctly aligned.

As noted above, the polymer in each of the stacked layers may comprise a polyaryletherketone. A polyaryletherketone may have repeating units of formula (I) below:

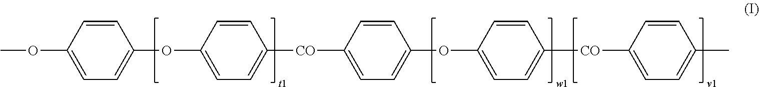

where t1 and w1 are independently represent 0 or 1 and v1 represents 0, 1 or 2.

The polyaryletherketone suitably includes at least 90, 95 or 99 mol % of repeat unit of formula I. The polyaryletherketone suitably includes at least 90, 95 or 99 weight % of repeat unit of formula I.

The polyaryletherketone may comprise or consist essentially of a repeat unit of formula I. Preferred polymeric materials comprise (or consist essentially of) a said repeat unit wherein t1=1, v1=0 and w1=0; t1=0, v1=0 and w1=0; t1=0, w1=1, v1=2; or t1=0, v1=1 and w1=0. More preferably, the polyaryletherketone comprises (e.g. consists essentially of) the repeat unit I, wherein t1=1, v1=0 and w1=0; or t1=0, v1=0 and w1=0. The most preferred polyaryletherketone comprises (especially consists essentially of) a said repeat unit wherein t1=1, v1=0 and w1=0.

The polyaryletherketone may suitably be selected from a group including polyetheretherketone, polyetherketone, polyetherketoneetherketoneketone and polyetherketoneketone. According to examples of the present invention, the polymer is particularly polyetheretherketone (PEEK).

The polyaryletherketone may have a Notched Izod Impact Strength (specimen 80 mm×10 mm×4 mm with a cut 0.25 mm notch (Type A), tested at 23° C., in accordance with ISO180) of at least 4 KJm⁻², preferably at least 5 KJm⁻², more preferably at least 6 KJm⁻². The Notched Izod Impact Strength, measured as mentioned above, may be less than 10 KJm⁻², suitably less than 8 KJm⁻². The Notched Izod Impact Strength, measured as mentioned above, may be at least 3 KJm⁻², suitably at least 4 KJm⁻², preferably at least 5 KJm⁻². The impact strength may be less than 50 KJm⁻², suitably less than 30 KJm⁻².

The polyaryletherketone (e.g. PEEK) suitably has a melt viscosity (MV) of at least 0.06 kNsm⁻², preferably has a MV of at least 0.09 kNsm⁻², more preferably at least 0.12 kNsm⁻². The polyaryletherketone (e.g. PEEK) may have a MV of less than 1.00 kNsm⁻² preferably less than 0.5 kNsm⁻².

The polyaryletherketone (e.g. PEEK) may have a MV in the range 0.09 to 0.5 kNsm⁻², preferably in the range 0.1 to 0.3 kNsm⁻², preferably having a MV in the range 0.1 to 0.2 kNsm⁻². An MV of 0.15 kNsm⁻² has been found to be particularly advantageous. MV is suitably measured using capillary rheometry operating at 400° C. at a shear rate of 1000 s⁻¹ using a tungsten carbide die, 0.5 mm×3.175 mm.

In a preferred embodiment, the polyaryletherketone (e.g. PEEK) has a melt viscosity (MV) of 0.09 kNsm⁻² to 0.5 kNsm⁻².

The polyaryletherketone may have a tensile strength, measured in accordance with ISO527 (specimen type 1 b) tested at 23° C. at a rate of 50 mm/minute of at least 20 MPa, preferably at least 60 MPa, more preferably at least 80 MPa. The tensile strength is preferably in the range 80-1 10 MPa, more preferably in the range 80-100 MPa.

The polyaryletherketone may have a flexural strength, measured in accordance with ISO178 (80 mm×10 mm×4 mm specimen, tested in three-point-bend at 23° C. at a rate of 2 mm/minute) of at least 50 MPa, preferably at least 100 MPa, more preferably at least 145 MPa. The flexural strength is preferably in the range 145-180 MPa, more preferably in the range 145-164 MPa. The polyaryletherketone may have a flexural modulus, measured in accordance with ISO178 (80 mm×10 mm×4 mm specimen, tested in three-point-bend at 23° C. at a rate of 2 mm/minute) of at least 1 GPa, suitably at least 2 GPa, preferably at least 3 GPa, more preferably at least 3.5 GPa. The flexural modulus is preferably in the range 3.5-4.5 GPa, more preferably in the range 3.5-4.1 GPa.

The polyaryletherketone may be amorphous or semi-crystalline. The polyaryletherketone is preferably crystallisable. The polyaryletherketone may be semi-crystalline. The level and extent of crystallinity in a polymer may be measured by wide angle X-ray diffraction (also referred to as Wide Angle X-ray Scattering or WAXS), for example as described by Blundell and Osborn (Polymer 24, 953, 1983). Alternatively, crystallinity may be assessed by Differential Scanning Calorimetry (DSC).

The level of crystallinity of said polyaryletherketone may be at least 1%, suitably at least 3%, preferably at least 5% and more preferably at least 10%. In especially preferred embodiments, the crystallinity may be greater than 25%. It may be less than 50% or less than 40%. The main peak of the melting endotherm (Tm) of said polyaryletherketone (if crystalline) may be at least 300° C.

The main peak of the melting endotherm (Tm) of the polyaryletherketone (if crystalline) may be at least 300° C. Where e.g. PEEK is used, the main peak of the melting endotherm (Tm) may be at least 300° C.

Where each layer comprises a composite material, the composite material may comprise any suitable amount of the polyaryletherketone (e.g. PEEK). For example, the composite material may comprise at least 20 volume %, preferably at least 25 volume %, more preferably at least 30 volume %, yet more preferably at least 35 volume %, even more preferably at least 37 volume % and most preferably at least 39 volume % polyaryletherketone (e.g. PEEK). The composite material comprises up to 48 volume % polyaryletherketone (e.g. PEEK). In some embodiments, the composite material may comprise up to 45 volume %, up to 43 volume % polyaryletherketone (e.g. PEEK).

In some embodiments, the composite material may comprise 20 to 48 volume %, preferably 30 to 48 volume %, more preferably 35 to 48 volume %, yet more preferably 37 to 48 volume % or 38 to 48 volume % polyaryletherketone (e.g. PEEK). More preferably, the composite material may comprise 39 to 48 volume %, even more preferably 39 to 45 volume % polyaryletherketone (e.g. PEEK). In some embodiments, the composite material may comprise 39 to 43 volume % polyaryletherketone (e.g. PEEK).

The composite material may additionally comprise of additives in the matrix. Particularly for medical applications, an example of additive includes bioactive agents, such as hydroxyapatite, or an image contrast agent, such as barium sulphate. In some examples, the composite material may comprise an image contrast agent. The image contrast agent may be present in all the layers or in selected layers of the composite material. The contrast agent may be an X-ray detectable material. For example, the contrast agent may be barium sulphate.

Any suitable reinforcement fibre may be used. The fibres used may be selected from inorganic or organic fibrous materials. The fibres may have a melting or decomposition temperature of greater than 200° C., for example, greater than 250° C. or greater than 300° C. In some embodiments, the fibres may have a melting temperature of greater than 350° C. or 500° C. Examples of suitable fibres include aramid fibres, carbon fibre, glass fibre, carbon fibre, silica fibre, zirconia fibre, silicon nitride fibre, boron fibre and potassium titanate fibre. Most preferred fibres are carbon fibres.

The volume ratio of reinforcement fibre to polyaryletherketone (e.g. PEEK) is 1.1:1 to 1.5:1, for example, 1.2:1 to 1:4:1.

The reinforcement fibre (e.g. carbon fibre) may have a tensile strength of greater than 4200 MPa, preferably greater than 4500 MPa, more preferably greater than 4800 MPa.

The reinforcement fibre (e.g. carbon fibre) may have a tensile modulus of greater than 200 GPa, preferably greater than 230 GPa, more preferably greater than 240 GPa.

The reinforcement fibre (e.g. carbon fibre) may have a strain at failure of greater than 1.1%, preferably, greater than 1.2%, 1.4% or 1.6% The reinforcement fibre (e.g. carbon fibre) may have a strain at failure of less than 2.2%, for instance, less than 2.0% or 1.9%.

In some embodiments, reinforcement fibre (e.g. carbon fibre) may have a strain at failure of 1.2 to 2.2%, for example, 1.4 to 2.0% or 1.6 to 1.9%. In one embodiment, the reinforcement fibre (e.g. carbon fibre) may have a strain at failure of 1.7 to 1.9%.

The reinforcement fibre (e.g. carbon fibre) may have a mass per unit length of 0.1 to 1.0 g/m, for example, 0.2 to 0.8 g/m. In some embodiments, the mass per unit length is 0.2 to 0.5 g/m.

The reinforcement fibre (e.g. carbon fibre) may have a density of greater than 1.65 g/cm³, preferably greater than 1.70 g/cm³. The reinforcement fibre (e.g. carbon fibre) may have a density of less than 1.85 g/cm³, preferably less than 1.80 g/cm³. In some embodiments, the reinforcement fibre (e.g. carbon fibre) may have a density of 1.70 to 1.85 g/cm³, for example, 1.75 to 1.80 g/cm³, or 1.78 to 1.79 g/cm³.

The reinforcement fibre (e.g. carbon fibre) may be provided in the form of a continuous tow. Any suitable tow size may be used. The tow size indicates the number of filaments in the tow. In some embodiments, the tow size may be 1000 to 24,000. In one embodiment, a tow size of 6000 to 12,000 may be employed.

Examples of suitable reinforcement fibre include carbon fibres supplied, for example, by Hexcel® under the trademark HexTow®.

The reinforcement fibre (e.g. carbon fibre) may be present in an amount of 30 to 68 volume %, preferably 40 to 65 volume %. Preferably, the reinforcement fibre may be present in an amount of 50 to 62 volume %, for instance, 52 to 58 volume % based on the total volume of the composite material.

The reinforcement fibre (e.g. carbon fibre) may be formed into filaments. Any suitable method may be employed. For example, the reinforcement fibres may be twisted or braided to form filaments. Where the composite material is formed into tape, the filaments may be substantially aligned along the longitudinal axis of the tape.

The amount of reinforcement fibre (e.g. carbon fibre) in the composite material can be controlled within a narrow range to enable the composite material to provide an optimised balance of mechanical properties.

In some embodiments, the composite material also comprises a contrast agent, e.g. barium sulphate. For example, barium sulphate may be present in the composite material in an amount of 2 to 20 weight % of the total weight of the composite material, for instance, 3 to 10 weight %. In a preferred embodiment, the amount of barium sulphate may be 4 to 8 weight %, more preferably 4 to 6 weight %. In a most preferred embodiment, the amount of barium sulphate may be 5 weight %.

By using controlled amounts of the reinforcement fibre (e.g. carbon fibre) in combination with contrast agent, e.g. barium sulphate, it may also be possible to vary the properties of the composite material in terms of its imageability under e.g. X-ray. For example, while barium sulphate may provide sufficient radio-opacity for an implantable device to be detected under e.g. X-ray, the amount of reinforcement fibre (e.g. carbon fibre) is controlled within narrow limits to provide or maintain sufficient translucency to allow the fracture in the underlying bone to be detected under imaging techniques e.g. X-ray.

Moreover, by using controlled amounts of the reinforcement fibre (e.g. carbon fibre) in combination with barium sulphate, the radio-translucency of the composite material may be optimized to reduce interference, such that dosing accuracy during radiotherapy can be maintained.

Any suitable contrast agent may be employed. Preferably, the contrast agent is detectable by X-ray. In some embodiments, the contrast agent comprises barium. For instance, the contrast agent may be barium sulphate.

Barium sulphate is a contrast medium that allows the composite material to be detected under imaging techniques, for example, X-ray. Accordingly, when the composite material is used in the manufacture of an implantable device, the device may be detected under e.g. X-ray.

The barium sulphate may have a D₁₀ particle size in the range of 0.1 to 1.0 microns; a D₅₀ particle size in the range of 0.5 to 2.0 microns and a Do particle size in the range of 1.0 to 5 microns. The D₁₀ particle size may be in the range of 0.1 to 0.6 microns, preferably 0.2 to 5 microns. The D₅₀ particle size may be in the range of 0.7 to 1.5 microns, preferably 0.8 to 1.3 microns. The Do particle size may be in the range of 1.5 to 3 microns, preferably in the range of 2.0 to 2.5 microns.

Suitable X-ray grade barium sulphate may be available from Merck-Millipore®.

Any suitable amount of contrast agent e.g. barium sulphate may be used. For example, contrast agent e.g. barium sulphate may be present in the composite material in an amount of 2 to 20 weight %, preferably, 3 to 15 weight %, for instance, 3 to 10 weight %. In a preferred embodiment, the amount of contrast agent e.g. barium sulphate may be 3 to 8 weight %, more preferably 3 to 5 or 4 to 6 weight %. In a most preferred embodiment, the amount of contrast agent e.g. barium sulphate may be 5 weight %.

The amount of contrast agent e.g. barium sulphate may be controlled, such that the radio-translucency of the composite material is optimized to reduce interference. This can allow dosing accuracy during radiotherapy to be maintained.

Furthermore, by controlling the relative amounts of the reinforcement fibre (e.g. carbon fibre) to barium sulphate, it may also be possible to vary the properties of the composite material in terms of its imageability under e.g. X-ray. For example, the relative amounts of the reinforcement fibre (e.g. carbon fibre) to barium sulphate may be controlled to allow the implantable device to be detected under e.g. X-ray, while maintaining sufficient translucency to allow the fracture in the underlying bone to be detected.

Moreover, the relative amounts of the reinforcement fibre (e.g. carbon fibre) to barium sulphate may be controlled, such that the radio-translucency of the composite material is optimized to reduce interference. This can allow dosing accuracy during radiotherapy to be maintained.

The composite material may be formed as tape. For example, the reinforcement fibre (e.g. carbon fibre) may be combined with the polyaryletherketone (e.g. PEEK) and formed into a tape. A plurality of tapes may be joined to form a layer and the layers compression moulded to form the compression moulded body portion of the device. In an embodiment, the polyaryletherketone (e.g. PEEK) may be heated to above its softening or melting temperature to melt or soften the polymer around the fibres to form the composite. The molten or soften polymer is then compressed around the fibres.

When heat is applied, suitable temperatures include temperatures of 320° C. and above, preferably, of 330° C. and above, more preferably, of 340° C. and above. In some embodiments, compression moulding may be carried out at temperatures of 320 to 450° C., preferably 330 to 400° C., more preferably 340 to 380° C. and yet more preferably 350 to 370° C. Suitably, pressures of at least 1.5 MPa or at least 2 MPa may be applied. Examples of suitable pressures range from 1.5 to 10 MPa, for instance, 2 to 8 MPa.

The tape or layer formed using the composite material of the present invention may have a thickness of 10 microns to 1 mm, preferably 100 to 300 microns, more preferably 140 to 200 microns.

Throughout this specification, the words “comprise” and “contain” and variations of them mean “including but not limited to”, and they are not intended to (and do not) exclude other components, integers or steps. Throughout this specification, the singular encompasses the plural unless the context otherwise requires. In particular, where the indefinite article is used, the specification is to be understood as contemplating plurality as well as singularity, unless the context requires otherwise. Throughout this specification, the term “about” is used to provide flexibility to a range endpoint by providing that a given value may be “a little above” or “a little below” the endpoint. The degree of flexibility of this term can be dictated by the particular variable and can be determined based on experience and the associated description herein.

Features, integers or characteristics described in conjunction with a particular aspect or example of the invention are to be understood to be applicable to any other aspect or example described herein unless incompatible therewith. All of the features disclosed in this specification, and/or all of the steps of any method or process so disclosed, may be combined in any combination, except combinations where at least some of such features and/or steps are mutually exclusive. The invention is not restricted to the details of any foregoing examples. The invention extends to any novel feature or combination of features disclosed in this specification. It will be also be appreciated that, throughout this specification, language in the general form of “X for Y” (where Y is some action, activity or step and X is some means for carrying out that action, activity or step) encompasses means X adapted or arranged specifically, but not exclusively, to do Y.

Each feature disclosed in this specification may be replaced by alternative features serving the same, equivalent or similar purpose, unless expressly stated otherwise. Thus, unless expressly stated otherwise, each feature disclosed is one example only of a generic series of equivalent or similar features.

The reader's attention is directed to all papers and documents which are filed concurrently with or previous to this specification in connection with this application and which are open to public inspection with this specification, and the contents of all such papers and documents are incorporated herein by reference.

1. A method of manufacturing a body, the method comprising:
forming a plurality of layers of composite material comprising reinforcement fibre and polyaryletherketone, a first layer of the plurality of layers being shaped such that in a first region it is narrower along a first axis than a second layer;
stacking the plurality of layers within a mould cavity of a mould tool such that the first layer defines a first side of the stack and the second layer is within the stack; and
compression moulding the stacked plurality of layers within the mould cavity;
wherein the mould cavity is defined by at least one surface of the mould tool shaped to align the slacked plurality of layers in the first region such that a resulting compression moulded body is thinner in the first region than in a second region.

2. A method according to claim 1, wherein the step of forming comprises cutting each of the plurality of layers to a predetermined shape having a first region, wherein in the first region the shape of at least some of the layers varies.

3. A method according to claim 1, further comprising arranging the stacked plurality of layers such that: in the first region layers deeper within the stack are wider along the first axis than layers closer to the first layer; the first region comprises a cut out portion of the first layer revealing at least part of the second layer; in the first region a subset of the plurality of stacked layers are shaped such that such that layers deeper within the stack are wider along the first axis than layers closer to the first layer; in the first region each layer is at least as wide along the first axis than an adjacent layer arranged closer to the first layer; or in the first region the stack comprises fewer layers than in the second region of the body.

4. A method of manufacturing according to claim 1, wherein the polyaryletherketone is polyetheretherketone (PEEK) and the reinforcement fibre is carbon fibre.

5. A method of manufacturing according to claim 4, wherein the carbon fibre is present in an amount of 30 to 68 volume %.

6. A method of manufacturing according to claim 1, wherein the composite material comprises a contrast agent in an amount 2 to 10 weight % of the total weight of the composite material.

7. A method of manufacturing according to claim 6, wherein the contrast agent is barium sulphate having a D₅₀ particle size in the range 0.5 to 2.0 microns.

8. A compression moulded body manufactured according to the method of claim 1, the compression moulded body comprising: a plurality of stacked layers of the composite material comprising reinforcement fibre and polyaryletherketone; wherein in a first region of the body a first layer defining a first side of the stack is narrower along a first axis than a second layer within the stack such that the body is thinner in the first region than in a second region.

9. A compression moulded body manufactured according to claim 8, wherein in the first region, layers deeper within the stack are wider along the first axis than layers closer to the first layer;
wherein the first region comprises a cut out portion of the first layer revealing at least part of the second layer;
wherein in the first region a subset of the plurality of stacked layers are shaped such that such that layers deeper within the stack are wider along the first axis than layers closer to the first layer;
wherein in the first region each layer is at least as wide along the first axis than an adjacent layer arranged closer to the first layer; or
wherein in the first region the stack comprises fewer layers than in the second region of the body.

10. A compression moulded body according to claim 9, wherein said body is a bone plate or an aircraft part.

11. A compression moulded body according to claim 10, wherein said body is a bone plate, and wherein the first region comprises a cut out such that the thickness of the plate varies within the first region.

12. A compression moulded body according to claim 11, wherein the cut out comprises a scalloped or chamfered region upon an edge of the bone plate, the cut out being less deep than the thickness of the bone plate in a second region and formed in a bone facing surface of the bone plate.

13. A compression moulded body according to claim 11, wherein the bone plate is elongated and a plurality of cut outs are provided along at least one long edge.

14. A compression moulded body according to claim 11, wherein each of the plurality of layers are curved along a length or width of the bone plate.

15. A compression moulded body comprising: a plurality of stacked layers of composite material comprising reinforcement fibre and polyaryletherketone; wherein in a first region of the body a first layer defining a first side of the stack is narrower along a first axis than a second layer within the stack such that the body is thinner in the first region than in a second region.

16. A compression moulded body as claimed in claim 15, wherein the compression moulded body is a bone plate.

17. A mould tool for forming a compression moulded body from a plurality of stacked layers of composite material comprising reinforcement fibre and polyaryletherketone, the mould tool comprising: a mould cavity configured to receive the stacked layers, a first layer defining a first side of the stack of the plurality of layers being shaped such that in a first region it is narrower along a first axis than a second layer within the stack;
wherein the mould cavity is defined by at least one surface of the mould tool shaped to align the slacked plurality of layers in the first region such that a resulting compression moulded body is thinner in the first region than in a second region.

18. A mould tool according to claim 15, wherein the mould tool comprises:
a first section having a mould opening defined by a side wall and configured to receive the stacked layers; and
a top section configured to close the mould opening to form a mould cavity;
wherein at least one of the first section and the top section is shaped to align the slacked plurality of layers in the first region.
